(19) United States
(12) Patent Application Publication
(10) Pub. No.: US 2025/0025421 A1
(43) Pub. Date: Jan. 23, 2025

(54) PREPARATION IN SOLID FORM COMPRISING ACTIVATED CARBON AND CHITOSAN, METHOD FOR PREPARING SAID PREPARATION, COMPOSITION COMPRISING SAID PREPARATION AND USES OF SAID COMPOSITION

(71) Applicant: S.I.I.T. S.R.L. - SERVIZIO INTERNAZIONALE IMBALLAGGI TERMOSALDANTI, Trezzano sul Naviglio (IT)

(72) Inventors: Andrea Domizio COSTA, Trezzano sul Naviglio (IT); Elena MADARO, Trezzano sul Naviglio (IT); Roberto MERICO, Trezzano sul Naviglio (IT)

(21) Appl. No.: 18/562,271

(22) PCT Filed: Jun. 10, 2022
(86) PCT No.: PCT/IB2022/055412

§ 371 Date: Nov. 17, 2023

(30) Foreign Application Priority Data
Jun. 10, 2021 (IT) 102021000015239

(51) Int. Cl. A61K 9/16; A61K 9/20; A61K 9/48; A61K 33/44; A61P 3/06

(52) U.S. Cl. CPC A61K 9/1652; A61K 9/205; A61K 9/4866; A61K 33/44; A61P 3/06

(57) ABSTRACT

A preparation in solid form comprising or, alternatively, consisting of: (i) an activated carbon; and (ii) a chitosan. preferably a plant chitosan from mushrooms, adsorbed onto said activated carbon. The chitosan has a deacety lation degree from 98% to 100% by weight. A method for preparing a preparation comprising the following steps: (I) preparing an aqueous solution of a chitosan, preferably plant chitosan from mushrooms, with a deacetylation degree from 98% to 100% by weight: (II) contacting the aqueous chitosan solution of step (I) with an activated carbon in powder form. to give a chitosan-impregnated activated carbon; (III) granulating the chitosan-impregnated activated carbon of step (II). to give a granulated impregnated activated carbon; (IV) drying the granulated impregnated activated carbon of step (III). to give said preparation. Said composition for use in the treatment of gastrointestinal disorders. Said composition for use in the treatment of hypercholesterolaemia.

The present invention relates to a preparation in solid form comprising or, alternatively, consisting of: (i) an activated carbon; and (ii) a chitosan, preferably a plant chitosan from mushrooms, absorbed on said activated carbon. The present invention also relates to a method for preparing a preparation comprising steps (I)-(IV).

The present invention also relates to a composition in solid form comprising said preparation or said preparation obtained by said method, and at least one physiologically and/or pharmaceutically acceptable excipient. The present invention also relates to said composition for use in a method of treatment, either preventive or curative, of a gastrointestinal disorder, or discomfort, or symptom, or disease associated with the presence or production of gas in the stomach and/or intestines of a subject.

The present invention also relates to said composition for use in a method of treatment, either preventive or curative, of a disorder, or discomfort, or a symptom, or a disease associated with hypercholesterolaemia in a subject.

It is estimated that, on average, an adult person has from 0.5 litres to 2.0 litres of gas in their digestive tract. These gases are mainly originated by three mechanisms, also concomitant, which are: aerophagy (or ingestion of air), ingestion of food containing gas or promoting gas development, and gas production by bacteria resident in the colon.

Aerophagia is a disorder of the digestive system characterised by a tendency to swallow air (e.g. during hasty swallowing of food and/or drink), which thus reaches the stomach instead of the lungs. Although aerophagia is—within certain limits—a physiological process, in some subjects this mechanism is particularly recurrent and takes on pathological contours.

Foods that can increase the amount of intestinal gas are legumes (e.g. beans, lentils, peas, chickpeas), vegetables belonging to the cabbage family (e.g. Brussels sprouts, broccoli, cabbage), sparkling drinks, and certain types of fruit (e.g. watermelon, melon, apples, avocado) especially when taken in the form of milkshakes.

Microorganisms resident in the colon ferment undigested or unabsorbed food residues, drawing energy from them and releasing gas. As a result, the greater the amount of unabsorbed or undigested substances in the colon, the greater the production of intestinal gas. As an example, in people intolerant to lactose sugar, unable to digest this sugar, the microflora develops large amounts of intestinal gas.

For the sake of completeness, as a fourth, statistically less recurrent mechanism, some drugs taken orally are gas generators. For example, sodium bicarbonate as an antacid—used to counteract stomach acidity—produces significant amounts of carbon dioxide.

The presence of gas in the stomach or intestines can give rise to a number of painful, uncomfortable or otherwise embarrassing disorders, diseases or symptoms, such as bloating in the abdomen, diarrhoea, excessive belching, meteorism and flatulence.

The remedies currently available to treat excessive gas in the digestive tract are natural (e.g. by taking carminative plants or their extracts, such as fennel, chamomile or ginger), re-educative chewing (to reduce aerophagia and facilitate digestive processes), dietary (by eating foods that favour the absorption of gas, or conversely by reducing or eliminating foods that favour the development of gas), or pharmacological.

The latter includes—inter alia—activated carbon (or active carbon), which is frequently used due to its considerable gas adsorbing capacity and its ability to exert a disinfectant action in the intestinal tract.

In this context, however, there is still a need for activated carbons that—for the same amount administered—are even more effective in the adsorption of gases and other substances, such as cholesterol, than commercially available activated carbons.

The Applicant, after long and intensive research and development activity, has developed a preparation, a composition and a method for preparing a preparation capable of increasing the adsorptive capacity of activated carbon. Said preparation, being based on activated carbon and chitosan, is stable over time and has no side effects or contraindications, particularly for paediatric or adolescent subjects. Moreover, said method is easily achievable, cost-effective, free of side effects, and extremely replicable.

Accordingly, it is an object of the present invention a preparation in solid form comprising or, alternatively, consisting of: (i) an activated carbon; and (ii) a chitosan, preferably a plant chitosan from mushrooms, adsorbed on said activated carbon, having the characteristics as defined in the accompanying claims.

It is also an object of the present invention a method of preparing a preparation comprising steps (I)-(IV), having the characteristics as defined in the accompanying claims.

It is further an object of the present invention a composition in solid form comprising said preparation or said preparation obtained by said method, and at least one physiologically and/or pharmaceutically acceptable excipient having the characteristics as defined in the accompanying claims.

It is also an object of the present invention a composition for use in a method of treatment, either preventive or curative, of a gastrointestinal disorder, or discomfort, or symptom, or disease associated with the presence or production of gas in the stomach and/or intestines of a subject., having the characteristics as defined in the accompanying claims.

It is also an object of the present invention a composition for use in a method of treatment, either preventive or curative, of a disorder, or discomfort, or a symptom, or a disease associated with hypercholesterolaemia in a subject, having the characteristics as defined in the accompanying claims.

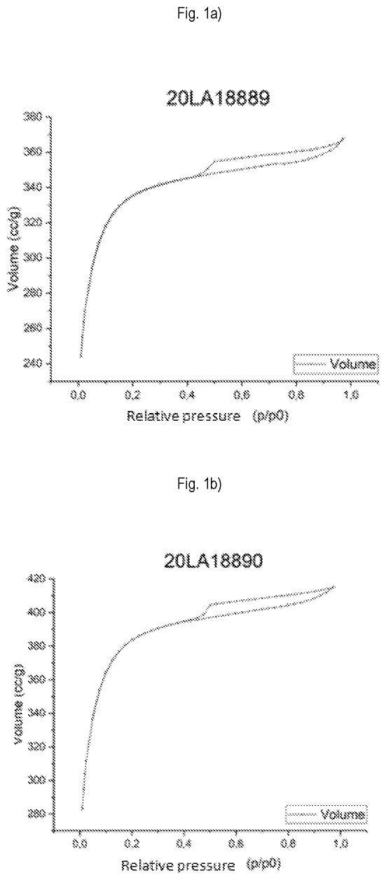

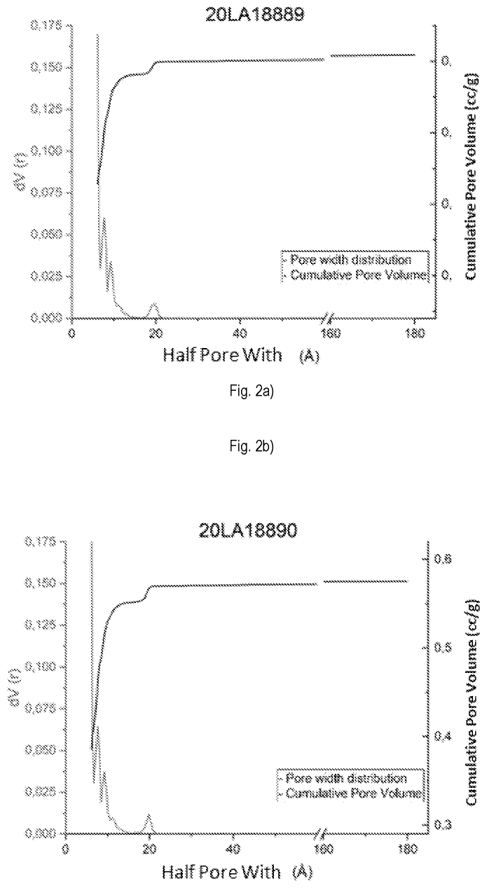

Preferred embodiments of the present invention will be described below by way of example, and therefore not limitative, with the help of the drawings wherein:
FIGS. 1a) and 1b) show two isotherms of the samples tested in Example 1, in each of which there is a hysteresis;
FIGS. 2a) and 2b) illustrate distributions of micropores and mesopores of the two samples tested in Example 1.

It is therefore an object of the present invention a preparation in solid form comprising or, alternatively, consisting of: (i) an activated carbon (or active carbon); and (ii) a chitosan, preferably a plant chitosan from mushrooms, adsorbed onto said activated carbon.

Preferably, said preparation is in granulate solid form.

More preferably, said granulate comprises granulate particles having an average particle size distribution such that from 85% to 100% by weight of said granulate, preferably from 90% to 99% by weight, passes through a sieve with a nominal aperture of 1.19 mm (US Sieve: 16 Mesh).

Preferably, said preparation in granulate solid form has the following characteristics:
loss on drying (LOD; measured at 70° C., on 2.7 grams of preparation; preferably using Mettler Toledo HB43-S Halogen as Halogen Moisture Analyser): from 1.50% to 2.50% by weight, preferably from 1.80% to 2.20% by weight, more preferably from 1.90% to 2.00% by weight, even more preferably about 1.96%; and/or
bulk density (measured by volumetric method, with a class A graduated cylinder, 50 ml capacity): from 0.23 g/cm³ to 0.38 g/cm³, preferably from 0.25 g/cm³ to 0.35 g/cm³, more preferably from 0.28 g/cm³ to 0.33 g/cm³, even more preferably about 0.31 g/cm ³.

The activated carbon used in the preparation of the present invention is preferably a activated carbon of plant origin. Said activated carbon of plant origin is preferably obtained by processing a wood powder and/or wood waste, subjected to combustion without flame (e.g. at temperatures from 500° C. to 600° C.), in the presence of sub-stechiometric amounts of oxygen. The resulting carbon is then chemically treated in the presence of dehydrating agents (such as phosphoric acid or zinc chloride), at temperatures from 400° C. to 1,000° C. After removal of the dehydrating agents by extraction, the carbon has a porous structure. Carbon with a porous structure is then activated with steam, and finally treated with acids to remove any impurities before being neutralised.

The activated carbon, preferably of plant origin, used in the preparation of the present invention preferably has one or more of the following characteristics:
adsorption power (phenazone; determined according to the protocol of the European Pharmacopoeia, 7th edition 2010 and BPC2009 (British Pharmacopoeia Commission) in the original version): ≥40 g/100 g; and/or
acid-soluble substances (determined according to the protocol of the European Pharmacopoeia, 7th edition 2010 and BPC2009 in the original version): ≤3% by weight; and/or
ethanol-soluble substances (determined according to the protocol of the European Pharmacopoeia, 7th edition 2010 and BPC2009 in the original version): ≤0.5% by weight.

As an example, an activated carbon that can be used in the present invention could be the product ‘Pulsorb® PGC’, manufactured and marketed by CHEMVIRON (Belgium; http://www.chemviron.eu).

As a further example, an activated carbon that can be used in the present invention could be product no. “1.02204”, brand name “Millipore”, manufactured and marketed by MERCK.

As a yet further example, an activated carbon that can be used in the present invention could be the compound with CAS No. 7440-44-0, having, for example, a molecular weight of about 12 g/mol.

Preferably, the activated carbon that can be used in the present invention has a bulk density (measured by volumetric method, with a class A graduated cylinder, 50 ml capacity) from 350 kg/m³ to 480 kg/m³, preferably from 380 kg/m³ to 450 kg/m³, even more preferably from 400 kg/m³ to 430 kg/m³.

Chitosan is a linear polymer of beta-(1,4)-D-glucosamine, a macromolecule with a partial positive charge used as an active component in mucolytic agents.

The chitosan used in the preparation of the present invention may be of animal origin or of plant origin, more preferably it is plant chitosan from mushrooms.

Said chitosan, preferably of plant origin from mushrooms, has an average molecular weight preferably from 1 kDa to 50 kDa, more preferably from 3 kDa to 30 kDa, even more preferably from 5 kDa to 25 kDa.

Said plant chitosan form mushrooms, present in the preparation of the present invention, has a chitosan fraction having an average molecular weight of from 3 kDa to 10 kDa, preferably from 5 kDa to 10 kDa, greater than the chitosan fraction having an average molecular weight from more than 10 kDa to 25 kDa, preferably from more than 10 kDa to 20 kDa,; moreover, said plant chitosan from mushrooms preferably has a weight ratio between the two fractions from 5:1 to 1:1, preferably from 4:1 to 1:1, or from 3:1 to 1:1, or from 2:1 to 1:1.

Said chitosan, preferably plant chitosan from mushrooms, has a deacetylation degree from 98% to 100% by weight, more preferably from 98.1% to 99% by weight, even more preferably from 98.2% to 98.5% by weight. “Deacetylation degree” of chitosan means the percentage of basic side groups that are not blocked by the presence of an acetyl group; thus, the greater the degree of deacetylation, the greater the percentage of free amine groups (—NH₂) and, therefore, the greater the solubility of chitosan in acidic or slightly acidic aqueous solutions. In acidic solution, said amine groups are converted into ammonium groups (—NH₃⁺) and thus the chitosan can interact with negatively charged sites on the surface and in the pores of the activated carbon and thus be strongly adsorbed. Without wishing to be limited to any scientific theory, it is believed that the adsorption of chitosan onto the surface of the activated carbon generates a molecular reorganisation of the solvent used (described in the method below) and the chitosan resulting in an increase in the adsorbent surface area and pore volume of the preparation.

An example of a chitosan of plant origin that can be used in the present invention could be a chitosan with a deacetylation degree ≥98%, a viscosity from 200 to 800 mpa·s (preferably about 600 mpa·s), and a pH value from 7 to 8. By way of example, a chitosan that can be used in the present invention could be the product “Chitosan”, marketed by Willows Ingredients Ltd (Ireland).

Said preparation has preferably:
specific surface area DFT (Density Functional Theory) according to ISO 15901:2007 from 980 m²/g to 1,300 m²/g, preferably from 1,020 m²/g to 1,200 m²/g, even more preferably from 1,050 m²/g to 1,150 m²/g; and/or
pore volume: from 0.580 cm³/g to 0.750 cm³/g, preferably from 0.590 cm³/g to 0.700 cm³/g, even more preferably from 0.620 cm³/g to 0.660 cm³/g.

More preferably, said preparation is characterised by the following parameters:
specific surface area BET (measured according to ISO 9277:2010, at −196° C. in the isothermal range from 0.01 to 0.15 relative pressure (w/wo): from 1,280 m²/g to 1,600 m²/g, preferably from 1,300 m²/g to 1,550 m²/g;
average pore size: from 1.60 nm to 2.00 nm, preferably from 1.75 nm to 1.95 nm;
micro-pore volume: from 0.520 cm³/g to 0.650 cm³/g, preferably from 0.550 cm³/g to 0.600 cm³/g.

Preferably, said preparation includes (amounts expressed with respect to the total weight of said preparation):
an amount of activated carbon, preferably activated carbon of vegetable origin, from 85% to 99% by weight, preferably from 90% to 98% by weight, even more preferably from 95% to 97% by weight; and
an amount of chitosan, preferably plant chitosan from mushrooms, more preferably plant chitosan from mushrooms with an average molecular weight from1 kDa to 50 kDa, from 1% to 15% by weight, preferably from 2% to 10% by weight, even more preferably from 3% to 5% by weight.

It is also an object of the present invention a method for preparing a preparation, preferably the preparation according to any of the above-described embodiments.

The method object of the present invention comprises the following steps:
(I) preparing an aqueous solution of a chitosan, preferably plant chitosan from mushrooms, having a deacetylation degree from 98% to 100% by weight;
(II) contacting the aqueous chitosan solution from step (I) with an activated carbon in powder form, to give a chitosan-impregnated activated carbon;
(III) granulating the chitosan-impregnated activated carbon of step (II), to give a granulated impregnated activated carbon;
(IV) drying the granulated impregnated activated carbon of step (III), to give said preparation.

Preferably, in step (I) of preparing the aqueous chitosan solution, a chitosan in powder form, preferably a plant chitosan from mushrooms in powder form, is added to a strong acid solution (preferably hydrochloric acid; more preferably with a molar concentration (M) 1 M, 2 M, 3 M, 4 M or 5 M) in water, wherein said strong acid solution in water has a pH value from 1 to 3 (preferably from 1 to 2, even more preferably from 1 to 1.5). The chitosan in powder form is added to the strong acid solution in the presence of mechanical stirring (preferably at a speed from 100 rpm to 300 rpm), until complete solubilisation of the chitosan powder, to give said aqueous solution of a chitosan used in step (II).

Preferably, the water used to prepare said strong acid solution is demineralised water. More preferably, the water used to prepare said strong acid solution is tap water, subjected to a first demineralisation step and a second, subsequent reverse osmosis step.

Step (I) is preferably conducted at room temperature (about 25° C.), and at atmospheric pressure (about 1 atm).

After preparation of the aqueous chitosan solution from step (I), said method comprises step (II) of contacting the aqueous chitosan solution with activated carbon in powder form.

The activated carbon used in step (II) of the present method is preferably an activated carbon in powder form, more preferably a plant activated carbon in powder form

The activated carbon in powder form used in (II) preferably comprises powder particles with an average particle size distribution-determined by laser beam diffraction analysis-such that an amount from 55% to 95% by volume, preferably from 65% to 85% by volume, has a size <45 μm.

Preferably, in step (II), the aqueous chitosan solution is poured, preferably flush poured, onto the activated carbon in powder form.

Preferably, in step (II), for every 100 parts by weight of activated carbon, from 65 to 90 parts by weight, preferably from 70 to 85 parts by weight, more preferably from 74 to 82 parts by weight, of aqueous chitosan solution are used.

Step (II) is preferably conducted at room temperature (about 25° C.), and at atmospheric pressure (about 1 atm). Step (II) is preferably conducted in air.

After step (II) of contacting the aqueous chitosan solution with the activated carbon in powder form, said method comprises step (III) of granulating the chitosan-impregnated activated carbon from step (II), to give the granulated impregnated activated carbon.

In the granulation step (III), a granulation device is used, e.g. a Diosna mod. P1/6 lab granulator. Preferably, step (II) and step (III) of the present method are at least partially contextual. This means that the aqueous chitosan solution of step (I) is contacted with, preferably poured (more preferably flush poured) onto, activated carbon in powder form pre-loaded in the granulation device having an impeller already activated in rotation, preferably at a speed from 50 rpm to 400 rpm, more preferably at a speed from 150 rpm to 250 rpm.

Preferably, after the addition of the aqueous solution of chitosan to the pre-loaded activated carbon in powder form has been completed, the impeller of the granulating device is kept activated in rotation, and a chopper of said granulating device is set at a speed from 700 rpm to 1,300 rpm, more preferably at a speed from 900 rpm to 1,100 rpm, for a time from 2 minutes to 15 minutes, more preferably from 3 minutes to 8 minutes.

Step (III) is preferably conducted at room temperature (about 25° C.), and at atmospheric pressure (about 1 atm).

Step (III) is preferably conducted in air.

At the end of step (III), the impregnated granulated activated carbon has the consistency of a slurry.

After step (III) of granulating the activated carbon, method includes step (IV) of drying the granulated impregnated activated carbon obtained from step (III), to give said preparation.

The drying step (IV) is preferably conducted at a temperature from 40° C. to 80° C., preferably from 50° C. to 70° C., even more preferably from 55° C. to 65° C., for a time from 6 hours to 16 hours, preferably from 7 hours to 14 hours, even more preferably from 8 hours to 12 hours.

It is also an object of the present invention a composition in solid form, preferably in tablets or capsules, comprising said preparation or the preparation obtained by said method, and at least one physiologically and/or pharmaceutically acceptable excipient.

Said composition in solid form preferably includes (the following amounts being expressed as a percentage of the individual component with respect to the total weight of the composition):
said preparation in an amount from 5% to 99.8%, preferably from 10% to 99.5%, even more preferably from 15% to 99%;
said at least one physiologically and/or pharmaceutically acceptable excipient in an amount from 95% to 0.2%, preferably from 90% to 0.5%, even more preferably from 85% to 1%.

Said at least one physiologically and/or pharmaceutically acceptable excipient is preferably selected from the group comprising or, alternatively, consisting of: a diluent, an anti-caking agent, a disintegrat, a binder, and mixtures thereof.

Said diluent is preferably included in said composition in an amount from 2% to 70% by weight, preferably from 10% to 60% by weight.

Preferably, said diluent is selected from maltodextrin, starch (preferably com starch), microcrystalline cellulose, sucrose, sorbitol, mannitol, xylitol, dibasic calcium phosphate, tribasic calcium phosphate, and mixtures thereof.

Said anti-caking agent is preferably included in said composition in an amount from 0.2% to 7% by weight, preferably from 0.5% to 5% by weight.

Preferably, said anti-caking agent is selected from magnesium stearate, glycerol behenate, talc, silicon salts (preferably amorphous silica), and mixtures thereof.

Said disintegrant is preferably included in said composition in an amount from 0.2% to 5% by weight, preferably from 0.5% to 4% by weight.

Preferably, said disintegrant is selected from sodium amidoglycolate, sodium carboxymethylcellulose, polyvinylpyrrolidone, and mixtures thereof.

Said binder is preferably included in said composition in an amount from 5% to 60% by weight, preferably from 10% to 45% by weight.

Preferably, said binder is selected from sucrose, dextrose, mannitol, sorbitol, fructose, microcrystalline cellulose, and mixtures thereof.

Said composition in solid form is preferably in tablet or capsule form.

Said composition in tablet solid form preferably includes:
said preparation in an amount from 5% to 40%, preferably from 10% to 30%, more preferably from 15% to 25%, even more preferably from 16% to 22%;
said at least one physiologically and/or pharmaceutically acceptable excipient in an amount from 95% to 60%, preferably from 90% to 70%, more preferably from 85% to 75%, even more preferably from 84% to 78%.

Said composition in tablet solid form most preferably includes:
said preparation in an amount from 5% to 40%, preferably from 10% to 30%, more preferably from 15% to 25%, even more preferably from 16% to 22%;
diluents, preferably starch (more preferably com starch) and/or maltodextrins, in an amount from 10% to 60%, more preferably from 35% to 55%, even more preferably from 40% to 50%;
anti-caking agents, preferably glycerol behenate, silicon salts (preferably amorphous silica) and/or magnesium stearate, in an amount from 0.5% to 5%, more preferably from 2% to 4.5%, even more preferably from 3% to 4%;
a disintegrant, preferably sodium carboxymethyl cellulose, in an amount from 0.5% to 4%, more preferably from 1% to 3%, even more preferably from 1.5% to 2.5%;
a binder, preferably microcrystalline cellulose, in an amount from 10% to 45%, more preferably from 20% to 40%, even more preferably from 25% to 35%.

Even more preferably, said composition in tablet solid form has the qualitative and quantitative composition in Table 1:

TABLE 1

| | mg/ | % by |
|---|---|---|
| Component | tablet | weight |
| PREPARATION | 112.50 | 18.75 |
| MICROC. CELLULOSE 200 E460 | 176.00 | 29.33 |
| CORN STARCH ID | 80.00 | 13.33 |
| MALTODEXTRIN IT19 (GLUCIDEX) USP | 200.00 | 33.33 |
| COMPRITOL AND ATO GLYCEROL BEHENATE | 12.00 | 2.00 |
| SYLOID 244FPNF ID (AMORPHOUS SILICA E551) | 1.50 | 0.25 |
| SODIUM CARBOXYMETHYLCELLULOSE - | 12.00 | 2.00 |
| DISOLCEL GF (SODIUM CROSCARMELLOSE) | | |
| PLANT MAGNESIUM STEARATE 765130 | 6.00 | 1.00 |
| LIGA E470B | | |
| Total | 600.00 | 100.00 |

Said composition in capsule solid form preferably includes:
said preparation in an amount form 70% to 99.8%, preferably from 75% to 99.5%, even more preferably from 80% to 99%;
said at least one physiologically and/or pharmaceutically acceptable excipient in an amount from 30% to 0.2%, preferably from 25% to 0.5%, even more preferably from 20% to 1%.

Said composition in capsule solid form most preferably includes:
said preparation in an amount from 70% to 99.8%, preferably from 75% to 99.5%, even more preferably from 80% to 99%;
an anti-caking agent, preferably magnesium stearate, in an amount from 0.1% to 5%, more preferably from 0.2% to 3%, even more preferably from 0.5% to 2%; or a diluent, preferably maltodextrin, in an amount from 3% to 25%, more preferably from 5% to 20%, even more preferably from 7% to 17%.

Even more preferably, said composition in capsule solid form has the qualitative and quantitative composition in Table 2 or Table 3:

TABLE 2

| Component | mg/capsule | % by weight |
|---|---|---|
| PREPARATION | 237 | 98.75 |
| PLANT MAGNESIUM STEARATE | 3 | 1.25 |
| VEG. 765130 LIGA E470B | | |
| Total | 240.00 | 100.00 |

TABLE 3

| | | % by |
|---|---|---|
| Component | mg/capsule | weight |
| PREPARATION | 210 | 87.50 |
| MALTODEXTRIN IT19 (GLUCIDEX) USP | 30 | 12.50 |
| Total | 240.00 | 100.00 |

It is also an object of the present invention said composition for use in a method of treatment, either preventive or curative, of a gastrointestinal disorder, or discomfort, or symptom, or disease associated with the presence or production of gas in the stomach and/or intestines of a subject.

The presence of polar functional groups (OH=hydroxyl and NH₂=amine), makes chitosan a molecule that can potentially be exploited as an adsorbent matrix. However, the physical and structural characteristics of chitosan (high crystallinity, low surface-to-volume ratio and limited mechanical strength) to date have limited its use as an adsorbent material. The adsorption of chitosan on activated carbon, obtained by the above-mentioned preparation method, results in a key structural change of the preparation object of the present invention because it allows the peculiarities of the individual compounds to be exploited efficiently and synergistically: the functional polar groups of chitosan and the physical and structural characteristics of activated carbon (high surface/volume ratio, high porosity, high mechanical strength).

Said gastrointestinal disorder, discomfort, symptom, or disease is preferably selected from the group comprising or, alternatively, consisting of: belching, flatulence, distension or bloating of the abdomen, abdominal pain, and meteorism.

Preferably, said composition for use is orally administered to said subject.

The dosage of administration of the composition in solid form of the invention to the subject will depend, for example, on: the condition to be treated, severity and course of the condition, previous therapy, sex and age of the patient, clinical picture of the subject.

The composition in solid form of the invention is administered to the subject one or more times a day during the treatment period; it may be administered as the sole treatment or in combination with other compositions or therapies (i.e. as an adjuvant) useful in the treatment, preventive and/or curative, of the gastrointestinal disorder, discomfort, symptom, or disease associated with the presence or production of gas in the stomach and/or intestines of a subject.

It is also an object of the present invention said composition for use in a method of treatment, either preventive or curative, of a disorder, or discomfort, or a symptom, or a disease associated with hypercholesterolaemia in a subject.

The concentration of cholesterol in the blood (cholesterolaemia) is defined as hypercholesterolaemia when cholesterolaemia levels are 240 mg/dl or more, for a subject who has been fasting for 12 hours. Low Density Lipoproteins (LDL) hypercholesterolaemia is considered one of the most important cardiovascular risk factors as it can cause serious diseases such as atherosclerosis.

The adsorption capacity of the preparation of said composition can also be exploited to adsorb cholesterol, preferably exogenous cholesterol, and thereby reduce its absorption and the occurrence of pathological conditions associated with hypercholesterolaemia.

Chitosan behaves as a polycationic (+) polymer that forms films with negatively charged surfaces. Negatively charged molecules in the gastrointestinal tract bind to the positively charged tertiary amine group (—NH³⁺) of chitosan. Thus, chitosan reduces the absorption of fats from the gastrointestinal tract by binding with the anionic carboxylic groups of fatty acids, interfering with the emulsification of neutral lipids (cholesterol and other sterols) by binding them with hydrophobic bonds.

Activated carbon is an adsorbent commonly used as drug, it is non-toxic and safe for use in oral administration because it cannot be digested in the gastrointestinal tract. Activated carbon is able to adsorb excess cholesterol in the intestine through binding with bile acids, effectively reducing their emulsifying activity and, consequently, the entry of cholesterol into the bloodstream.

In view of the above, the preparation object of the present invention combines the cholesterol-lowering properties of the individual molecules in a single complex in a synergistic and complementary manner. The increase in surface area also confers a greater adsorptive capacity than that of the individual molecules.

Said disorder, discomfort, symptom, or disease associated with hypercholesterolaemia is preferably selected from the group comprising or, alternatively, consisting of: atherosclerosis, thrombosis, stroke, heart attack, and angina. Preferably, said composition for use is orally administered to said subject.

The dosage of administration of the composition in solid form of the invention to the subject will depend, for example, on: the condition to be treated, severity and course of the condition, previous therapy, sex and age of the patient, clinical picture of the subject.

The composition in solid form of the invention is administered to the subject one or more times a day during the treatment period; it may be administered as the sole treatment or in combination with other compositions or therapies (i.e., as an adjuvant) useful in the treatment, preventive and/or curative, of the disorder, discomfort, symptom, or disease associated with hypercholesterolaemia.

Embodiments FRn object of the present invention are as follows.

FR1. A preparation in solid form comprising or, alternatively, consisting of: (i) an activated carbon; and (ii) a chitosan, preferably a plant chitosan from mushrooms, adsorbed onto said activated carbon; wherein said chitosan has a deacetylation degree from 98% to 100% by weight.

FR2. The preparation according to FR1, wherein said preparation has:
specific surface area DFT (Density Functional Theory) according to ISO 15901:2007 from 980 m²/g to 1,300 m²/g, preferably from 1,020 m²/g to 1,200 m²/g, even more preferably from 1,050 m²/g to 1,150 m²/g; and
pore volume: from 0.580 cm³/g to 0.750 cm³/g, preferably from 0.590 cm³/g to 0.700 cm³/g, even more preferably from 0.620 cm³/g to 0.660 cm³/g.

FR3. The preparation according to any one of FR1-2, wherein said preparation comprises (amounts expressed with respect to the total weight of said preparation):
an amount of activated carbon, preferably activated carbon of plant origin, from 85% to 99% by weight, preferably from 90% to 98% by weight, even more preferably from 95% to 97% by weight; and
an amount of chitosan, preferably plant chitosan from mushrooms with an average molecular weight from 1 kDa to 50 kDa, from 1% to 15% by weight, preferably from 2% to 10% by weight, even more preferably from 3% to 5% by weight.

FR4. The preparation according to any one of FR1-3, wherein said preparation is in granulate solid form, wherein said granulate comprises granulate particles having an average particle size distribution such that an amount from 85% to 100% by weight, preferably from 90% to 99% by weight, passes through a sieve with a nominal aperture of 1.19 mm.

FR5. A method for preparing a preparation according to any one of FR1-4, said method comprising the following steps:
(I) preparing an aqueous solution of a chitosan, preferably plant chitosan from mushrooms, with a deacetylation degree from 98% to 100% by weight;
(II) contacting the aqueous chitosan solution of step (I) with an activated carbon in powder form, to give a chitosan-impregnated activated carbon;
(III) granulating the chitosan-impregnated activated carbon of step (II), to give a granulated impregnated activated carbon;
(IV) drying the granulated impregnated activated carbon of step (III), to give said preparation.

FR6. The preparation method according to FR5, wherein:
activated carbon in powder form of step (II), preferably an activated carbon of plant origin, comprises powder particles with an average particle size distribution-determined by laser beam diffraction analysis-such that from 55% to 95% by volume, preferably from 65% to 85% by volume, have a size <45 μm; and/or
steps (II) and (III) are at least partially contextual, where the aqueous chitosan solution of step (I) is contacted with activated carbon in powder form pre-loaded in a granulation device equipped with an impeller that has already been activated in rotation.

FR7. A composition in solid form comprising the preparation according to any one of FR1-4, or the preparation obtained by the method according to any one of FR5-6, and at least one physiologically and/or pharmaceutically acceptable excipient; wherein:
said composition is in tablet solid form and includes:
   said preparation in an amount from 5% to 40%, preferably from 10% to 30%, more preferably from 15% to 25%, even more preferably from 16% to 22%;
   diluents, preferably starch (more preferably com starch) and/or maltodextrins, in an amount from 10% to 60%, more preferably from 35% to 55%, even more preferably from 40% to 50%;
   anti-caking agents, preferably glycerol behenate, silicon salts (preferably amorphous silica) and/or magnesium stearate, in an amount from 0.5% to 5%, more preferably from 2% to 4.5%, even more preferably from 3% to 4%;
   a disintegrant, preferably sodium carboxymethyl cellulose, in an amount from 0.5% to 4%, more preferably from 1% to 3%, even more preferably from 1.5% to 2.5%; and
   a binder, preferably microcrystalline cellulose, in an amount from 10% to 45%, more preferably from 20% to 40%, even more preferably from 25% to 35%; or said composition is in capsule solid form and includes:
   said preparation in an amount from 70% to 99.8%, preferably from 75% to 99.5%, even more preferably from 80% to 99%;
   an anti-caking agent, preferably magnesium stearate, in an amount from 0.1% to 5%, more preferably from 0.2% to 3%, even more preferably from 0.5% to 2%; or a diluent, preferably maltodextrin, in an amount from 3% to 25%, more preferably from 5% to 20%, even more preferably from 7% to 17%.

FR8. The composition according to FR7, wherein said composition is for use in a method of treatment, preventative or curative, of a gastrointestinal disorder, or discomfort, or symptom, or disease associated with the presence or production of gas in the stomach and/or intestines of a subject; preferably said composition being orally administered to said subject.

FR9. The composition for use according to FR8, wherein said gastrointestinal disorder, discomfort, symptom, or disease is selected from the group comprising or, alternatively, consisting of: belching, flatulence, distension or swelling of the abdomen, abdominal pain, and meteorism.

FR10. The composition according to FR7, wherein said composition is for use in a method of treatment, either preventive or curative, of a disorder, or discomfort, or a symptom, or a disease associated with hypercholesterolaemia in a subject.

The following are examples, given by way of example and not being a limitation of the present invention.

### EXAMPLES.

Example 1: Evaluation of specific surface area by BET method and pore size distribution by DFT.
The following two powder samples were analysed:
   20LA18889: activated carbon;
   20LA18890: preparation of the present invention (activated carbon+plant chitosan from mushrooms adsorbed on said activated carbon).

### Analytical Method:

Prior to analysis, the samples were degassed for at least 16 hours at 200° C. to completely remove the adsorbed moisture. The entire isotherm was collected to assess the specific surface area, as well as the pore size and pore volume of the samples. The specific surface area (SSA) was determined using the Brunauer-Emmett-Teller (BET) method according to ISO 9277:2010. The SSA was then determined at −196° C. in the isothermal range from 0.01 to 0.15 relative pressure (w/w₀). All measurements were performed using an Autosorb iQ2 (Quantachrome) instrument, using nitrogen gas. The pore size distribution was determined using Density Functional Theory (DFT) according to ISO 15901:2007. The calculation model used in the analysis is the nitrogen adsorbed on carbon (77 K) assuming pores in slit or slit-like shape. The pore volumes were evaluated at the last adsorption points of the isotherm (w/p₀≈0.975). The average pore size is expressed as diameter. The specific surface area and micropore volume were also calculated using the DFT method.

The isotherm of both samples exhibits hysteresis, as shown in FIG. 1a) and FIG. 1b), resulting from condensation in the pores. The distributions of micropores and mesopores are shown in FIG. 2a) and FIG. 2b).

The instrumental parameters of the SSA measurements for samples 20LA18889 and 20LA18890 are shown in Table 4 and Table 5 below, respectively:

TABLE 4

| Sample weight | 0.2578 | g |
|---|---|---|
| Approximate degassing time (outgas) | 7.5 | hours |
| Analysis gas | Nitrogen | |
| Analysis time | 9:43 hrs:min | |
| Analysis mode | Standard | |
| VoidVol mode | He measurement | |
| Instrument | Autosorb iQ Station 1 | |
| Final degassing temperature (outgas) | 250° | C. |
| Non-ideality | 6.58e−05 1/Torr | |
| Bath temperature | 73.35 | K |
| Cold Zone V | 5.87582 | cc |
| Extended information | Available | |
| Cell type | 9 | mm |
| Re-measurement of VoidVol | Off | |
| Hot zone V | 7.55413 | cc |

TABLE 5

| Sample weight | 0.2463 | g |
|---|---|---|
| Approximate degassing time (outgas) | 7.5 | hours |
| Analysis gas | Nitrogen | |
| Analysis time | 10:52 hrs:min | |
| Analysis mode | Standard | |
| VoidVol mode | He measurement | |
| Instrument | Autosorb iQ Station 2 | |
| Final degassing temperature (outgas) | 250° | C. |
| Non-ideality | 6.58e−05 1/Torr | |
| Bath temperature | 73.35 | K |
| Cold Zone V | 5.80009 | cc |
| Extended information | Available | |
| Cell type | 9 | mm |
| Re-measurement of VoidVol | Off | |
| Hot zone V | 9.19707 | cc |

SSA, pore volume, average pore size and micropore volume obtained using the DFT method are shown in Table 6 below:

TABLE 6

| | Activated | Carbon + chitosan |
|---|---|---|
| | carbon | preparation |
| Parameters | (20LA18889) | (20LA18890) |
| BET specific surface area (m²/g) ± | 1244 ± 95 | 1425 ± 126 |
| expanded uncertainty (k = 2) | | |
| Specific surface area DFT (m²/g) | 952 | 1103 |
| Pore volume (cm³/g) | 0.569 | 0.642 |
| Average pore size (nm) | 1.83 | 1.81 |
| Micropore volume (cm³/g) | 0.509 | 0.575 |

### Conclusions:

The samples are characterised by a high specific surface area (SSA), with the preparation object of the present invention (20LA18890) characterised by SSA-DFT that is 15% higher than activated carbon alone (20LA18889) without adsorbed chitosan. Both samples are highly microporous. According to the IUPAC classification (THOMMES, Matthias, et al. Physisorption of gases, with special reference to the evaluation of surface area and pore size distribution (IUPAC Technical Report). Pure and Applied Chemistry, 2015, 87.9-10:1060-1061) both samples exhibit mesopores and micropores as they are characterised by a type Il isotherm and a type H4 hysteresis.

### Experimental Part

Evaluation of pore size distribution and specific surface area by nitrogen physio-absorption and using DFT and BET methods

1. Aim

Evaluation of the pore size distribution using the DFT method and analysis of the specific surface area using the BET method of three samples.

2. Description of Samples The evaluation was conducted on 3 powder samples. The entry codes and sample descriptions are given in Table 7.

TABLE 7
Codes and descriptions of test samples

| Entry code | Description |
|---|---|
| 21LA24480 | Carbon Tablet |
| | Batch: NU626 |
| | S.I.I.T. Srl |
| | 17 Dec. 2021 |
| 21LA24481 | Adsorbix 100 mg Tablet |
| | Batch: PS243-1 |
| | S.I.I.T. Srl |
| | 17 Dec. 2021 |
| 21LA24482 | Adsorbix 300 mg Tablet |
| | Batch: PR281-8 |
| | S.I.I.T. Srl |
| | 17 Dec. 2021 |

3. Analytical method

Before analysis, the three samples were degassed for at least 16 hours at 100° C. to completely remove the adsorbed moisture.

The entire isotherm was collected to assess the pore size and volume of the samples.

The pore size distribution was determined using the density functional theory (DFT) method according to ISO 15901:2007. The calculation model chosen is the nitrogen adsorbed on carbon (77 K) assuming slit-like pores.

Pore volume was evaluated using the last adsorption points of the isotherm (p/p0≈0.975), while average pore size was calculated as average pore diameter.

The specific surface area (SSA) was determined using the Brunauer-Emmett-Teller (BET) method according to ISO 9277:2010. The SSA was determined at −196° C. in the isothermal range of 0.005 to 0.3 relative pressure (w/p0), depending on the samples.

All measurements were performed with Autosorb iQ2 (Quantachrome), using nitrogen as the gas.

4. Results

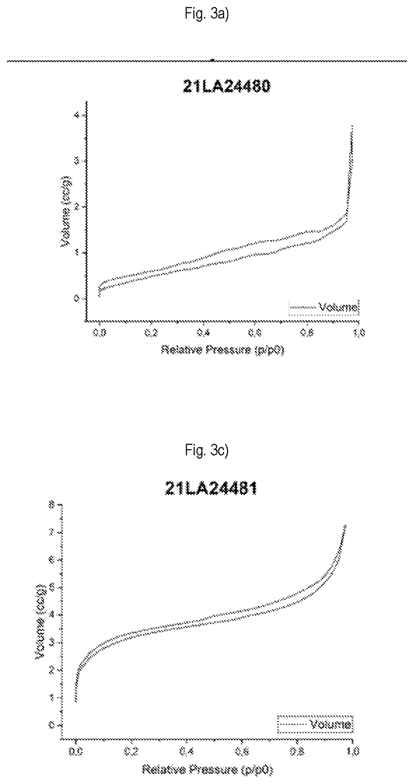

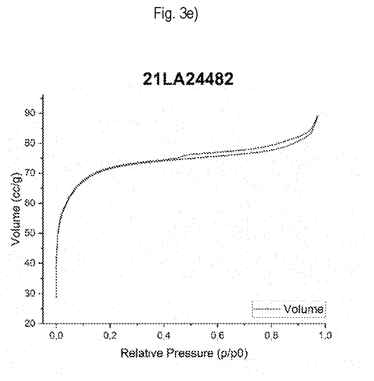

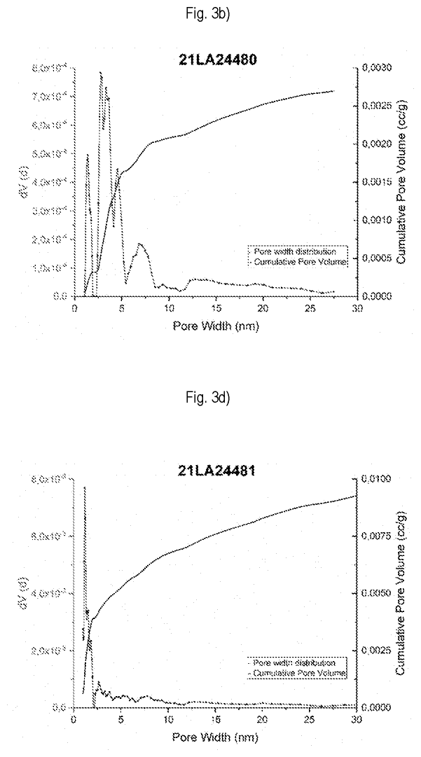

The sample isotherms are characterised by hysteresis due to nitrogen pore condensation (FIG. 3 a-c-e).

FIG. 3a refers to the isotherm values of sample 21LA24480.

FIG. 3c refers to the isotherm values of sample 21LA24481.

FIG. 3e refers to the isotherm values of sample 21LA24482.

The SSA values, pore volumes, average pore size (pore diameter) and micropore volumes obtained by the DFT method are shown in Table 8.

micropore volumes obtained by the DFT method are shown in Table 8.The distributions of micropores and mesopores are shown in FIG. 3b-d-f.

TABLE 8
Sample characteristics and values.

| Characteristics | 21LA24480 | 21LA24481 | 21LA24482 |
|---|---|---|---|
| BET surface area | 2.11 ± 0.16 | 11.52 ± 0.87 | 268 ± 27 |
| (m²/g) ± expanded | | | |
| uncertainty (k = 2) | | | |
| Pore volume | 0.0046 | 0.011 | 0.14 |
| (cm³)/g) | | | |
| Average pore size | 8.8 | 3.9 | 2.1 |
| (nm) | | | |
| Micropore volume | 0.003 | 0.01 | 0.12 |
| (cm³/g) | | | |

5. Conclusions

All samples were identified as porous materials. In particular:
Sample 21LA24480 is mainly mesoporous as the average pore size is more than 2 nm1 (i.e. 8.8 nm);
Sample 21LA24481 shows a significant increase in micropores, although it is mesoporous due to its average pore size of 3.9 nm;
sample 21LA24482 is mainly microporous, as the average pore size is about 2 nm (i.e. 2.1 nm).

### Experimental Report

### Evaluation of the cholesterol absorption capacity of four formulations

### AIM

This study aimed to evaluate the cholesterol absorption activity of four formulations under gastrointestinal conditions. All experiments were performed at the ECSIN-ECAMRICERT SRL Laboratory, Corso Stati Uniti, Laboratorio, Corso Stati Uniti, 4-35127 Padua, Italy.

### Materials and Methods

### Samples

The evaluation of cholesterol absorption capacity under gastrointestinal conditions was performed on the four formulations described in Table 9.

TABLE 9

| | Reception | |
|---|---|---|
| Sample | Number | Batch |
| CHITOSAN | 22LA02592 | 2104590 |
| cod. 01002945 | | |
| (CHITO) | | |
| PLANT CARBON | 22LA02593 | 2111686 |
| cod. 01002483 | | |
| (CARB) | | |
| ADSORBIX 300 MG | 22LA02594 | PR281-8(A) |
| (ADSO) | | |
| LIPOLID X-PLUS | 22LA02595 | VV132 |
| (KAPTUFAT) | | |
| (LIPO) | | |

Determination of Cholesterol Absorption Activity of Formulations

### Methanol

A 20 g/L concentration of the four formulations (CHITOSAN, PLANT CARBON, ADSORBIX 300 MG and LIPOLID X-PLUS) was added to freshly prepared 50 mg/L cholesterol-containing methanol and the suspensions were kept under constant stirring (100 rpm) for 2 hours at 37° C. After 120 minutes of incubation, aliquots were collected, centrifuged at 12,000 rpm for 30 minutes at 25° C. and supernatants were stored at −20° C. Cholesterol quantification was performed with a commercial kit (Sigma-Aldrich), according to the manufacturer's instructions. The results are expressed as: (i) percentage of cholesterol recovery in the supernatants compared to the initial cholesterol content; and (ii) percentage of reduction compared to non-methanol-treated cholesterol.

### Gastrointestinal mimetic solution (GIMS)

The cholesterol adsorption capacity of the formulations was evaluated according to the protocol described by Inanan and colleagues (Inanan, T., Tüzmen, N., Akgöl, S., & Denizli, A. (2016). Selective adsorption of cholesterol by molecularly imprinted polymeric nanospheres and application to GIMS. International Journal of Biological Macromolecules, 92, 451-460), with minor modifications. Briefly, the gastrointestinal mimetic solution was prepared by dissolving sodium deoxycholate and sodium cholate, used as bile salts to simulate the emulsifying activity of bile, in a mixture in a solution consisting of KH2PO4, NaOH and distilled water.

The pH was adjusted to 7.5 and nitrogen gas was passed through the solution for 15-20 minutes to simulate the predominantly anoxic condition of the gastrointestinal tract and limit cholesterol oxidation.

Cholesterol stock solution (4X) was prepared by resuspending cholesterol in GIMS at a concentration of 200 mg/L, and sonicated for at least 30 minutes at 40° C.

A 20 g/L concentration of the four formulations was added to the 50 mg/L cholesterol-containing GIMS solution, and the suspensions were kept under constant stirring (100 rpm) for 2 h at 37° C. At selected time intervals (0, 15, 30, 60 and 120 min), aliquots were collected, centrifuged at 12,000 rpm for 30 min at room temperature and the supernatants were stored at −20° C.

Cholesterol quantification was performed with a commercial kit (Sigma-Aldrich), according to the manufacturer's instructions. Since interference was observed with the cholesterol quantification kit for LIPOLID X-PLUS, for this formulation the quantification of cholesterol in the supernatants was determined by Gas Chromatography-Mass Spectrometry (GC-MS).

The results are expressed as: (i) percentage of cholesterol recovery in the supernatants compared to the initial cholesterol content; and (ii) percentage of reduction compared to untreated GIMS containing cholesterol.

### Statistical Analysis

All data are presented as mean±standard deviation (SD) of three independent experiments. To determine whether there were statistically significant differences between the treatments, a t-test analysis was performed. T-test is a statistical method used to test for differences between two averages. Differences between groups were considered significant at p<0.05. All statistical analyses were performed with OriginLab software.

### RESULTS

Cholesterol is an essential component of the human body, as it stabilises cell membranes and serves as a precursor for bile acids, vitamin D and steroid hormones. The plasma concentration of cholesterol (i.e. cholesterolaemia) is regulated by endogenous and exogenous pathways of cholesterol metabolism.

In the endogenous pathway, cholesterol is synthesised by the liver and extrahepatic tissues and may enter the blood circulation as a component of lipoproteins or be secreted into the bile. This pathway is not sufficient to ensure proper cholesterol homeostasis and the cholesterol pool must be constantly replenished through the absorption of cholesterol from bile and food in the intestine, in a process known as the exogenous cholesterol pathway. However, due to the high cholesterol content in the diet of Western countries, cholesterol absorbed via the exogenous pathway tends to be excessive, leading to a significant increase in plasma cholesterol concentration (i.e. hyperlipidaemia). Hyperlipidaemia is known to be a key point in the development of cardiovascular disease, particularly in the formation of atherosclerotic plaques, potentially leading to heart failure and stroke. To reduce excess dietary intake of cholesterol, one potential solution lies in the removal of cholesterol during gastrointestinal digestion.

In the present work, the cholesterol adsorption capacity of the four formulations was evaluated from gastrointestinal fluids (GIMS).

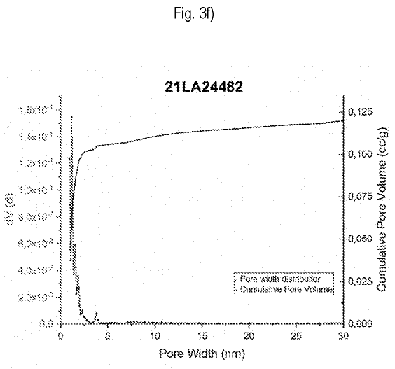

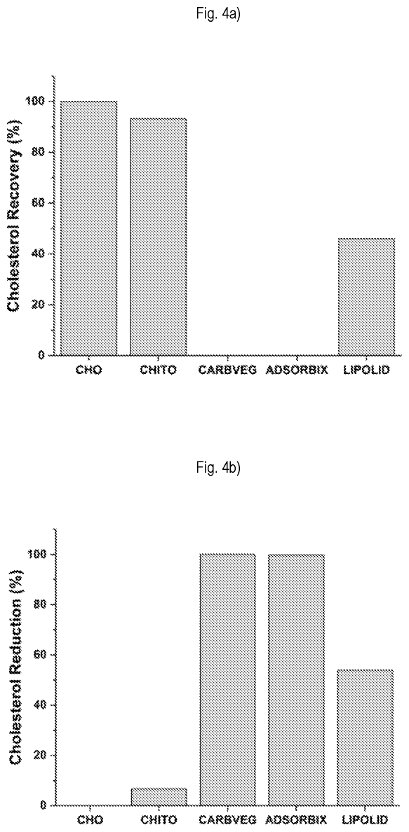

Before conducting the activity in GIMS, the capacity of preparatins to adsorb cholesterol was evaluated in methanol. The latter is a solvent in which the dissolution of cholesterol is complete, representing an easier system to explore the cholesterol adsorption activity of formulations. With the exception of CHITO, the formulations under investigation efficiently absorb methanol cholesterol, as indicated by cholesterol recovery (FIG. 4A) and cholesterol reduction (FIG. 4B). In particular, ABSORBIX and CARBVEG are able to completely remove cholesterol dissolved in methanol (about 100% cholesterol adsorption for both ADSORBIX and CARBVEG) (FIG. 4A and 4B).

FIG. 4. Cholesterol recovery (A) and cholesterol reduction (B), expressed as a percentage (%) relative to the initial cholesterol content and untreated cholesterol-containing methanol, respectively, obtained from the four formulations after 120 minutes of incubation.

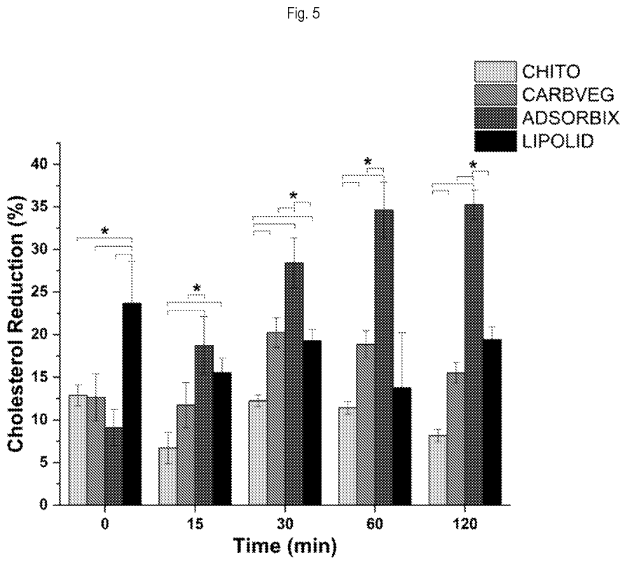

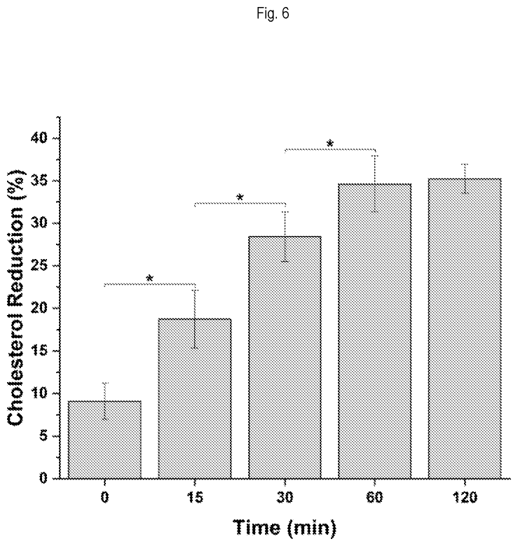

Evaluation of cholesterol adsorption in GIMS according to formulation shows a time-dependent cholesterol reduction trend for CHITO and LIPOLID (FIG. 5 and Table 10). Both formulations reach maximum cholesterol reduction (about 12% and 20% for CHITO and LIPOLID respectively) once added to the cholesterol-containing GIM (0 min), which probably indicates a rapid Cholesterol absorption followed by saturation. At 0 minutes, cholesterol reduction with LIPOLID is significantly greater than with the other formulations tested (FIG. 5 and Table 10). A clear time-dependent cholesterol reduction trend was shown for the ADSORBIX formulation, which shows a significant increase in cholesterol reduction over time, reaching the maximum cholesterol reduction after 60 minutes of incubation (FIG. 5, FIG. 6 and Table 10). Furthermore, ADSORBIX shows a significantly higher cholesterol reduction capacity than the other formulations tested from 30 minutes onwards (FIG. 5). Finally, CARBVEG achieves maximum cholesterol reduction (about 20%) at 30 minutes, after which no significant changes are observed.

FIG. 5. Cholesterol reduction obtained with the four formulations (CHITO, CARBVEG, ADSORBIX, LIPOLID) at different times (0, 15, 30, 60 and 120 min). Cholesterol reduction is expressed as a percentage (%) compared to the untreated GIMS containing cholesterol. Results are reported as mean +standard deviation. * p<0.05.

| Time | Cholesterol Recovery (%) | | | | Cholesterol Reduction (%) | | | |
|---|---|---|---|---|---|---|---|---|
| (min) | CHITO | CARBVEG | ADSORBIX | LIPOLID | CHITO | CARBVEG | ADSORBIX | LIPOLID |
| 0 | 87.1 ± 1.2 | 87.3 ± 2.8 | 90.9 ± 2.1 | 76.3 ± 4.9 | 12.9 ± 1.2 | 12.7 ± 2.8 | 9.1 ± 2.1 | 23.7 ± 4.9 |
| 15 | 93.3 ± 1.9 | 88.3 ± 2.6 | 81.3 ± 3.4 | 84.5 ± 1.7 | 6.7 ± 1.9 | 11.7 ± 2.6 | 18.7 ± 3.4 | 15.5 ± 1.7 |
| 30 | 87.8 ± 0.7 | 79.8 ± 1.7 | 71.6 ± 2.9 | 80.7 ± 1.3 | 12.2 ± 0.7 | 20.2 ± 1.7 | 28.4 ± 2.9 | 19.3 ± 1.3 |
| 60 | 88.6 ± 0.7 | 81.1 ± 1.6 | 65.4 ± 3.3 | 86.2 ± 6.4 | 11.4 ± 0.8 | 18.9 ± 1.6 | 34.6 ± 3.3 | 13.8 ± 6.3 |
| 120 | 91.8 ± 0.8 | 84.5 ± 1.2 | 64.8 ± 1.7 | 80.6 ± 1.5 | 8.2 ± 0.8 | 15.5 ± 1.2 | 35.2 ± 1.8 | 19.4 ± 1.5 |

Table 10. Recovery and reduction of cholesterol in supernatants following exposure of cholesterol-containing GIMS to the four formulations (CHITO, CARBVEG, ADSORBIX, LIPOLID) at different times (0, 15, 30, 60 and 120 minutes). Cholesterol recovery and reduction are expressed as a percentage (%) compared to untreated GIMS containing cholesterol. FIG. 6. Cholesterol reduction trend in cholesterol-containing GIMS following exposure to ADSORBIX at different times (0, 15, 30, 60 and 1210 minutes). Results are reported as mean +standard deviation. * p<0.05.

### Conclusions

In the light of the results obtained, among the formulations tested (CHITOSAN, PLANT CARBON, ADSORBIX 300 MG and LIPOLID X-PLUS), ADSORBIX 300 MG shows the highest cholesterol-lowering capacity and saturation time. Accordingly, it is able to maintain its cholesterol-eliminating capacity for longer.

TABLE 11
Adsorbix 300 is in the form of 1500 mg tablets to be administered
once a day and contains 300 mg of plant carbon, e.g.
Pulsorb PGC type, and 12 mg of mushroom chitosan, e.g.
80 mesh 98% DA, and carrier and excipients.

Product: Adsorbix 300
Batch: PR281-8(A)
Formulation type: TABLETS
Net weight (g): 1.5
Daily dose: 1

Trade name
PLANT CARBON PULSORB PGC
CHITOSAN FROM MUSHROOM 80 MESH 98% DA
Hydrochloric acid 1N
DEMINERALISED WATER
MICROC. CELLULOSE 102 E460
CALCIUM CARBONATE GRAN.ID PHARMAGNESIA CC TYPE 140
MALTODEXTRINS (GLUCIDEX 9) ID
SYLOID 244FPNF ID (AMORPHOUS SILICA E551)
KLUCEL NUTRA D
VEG. MAGNESIUM STEARATE 765130 LIGA E470B
POLYPLASDONE XL

TABLE 12
Adsorbix 100 is in the form of 950 g tablets to be administered
once a day and contains 300 mg plant carbon, e.g. Pulsorb
PGC type, and 4 mg chitosan from mushrooms, e.g. 80 mesh
98% DA, and carrier and excipients.

Product: Adsorbix 100 mg
Client: SIIT
Batch: PS243-1
Formulation type: TABLETS
Net weight (g): 0.95
Daily dose: 1

Trade name
PLANT CARBON PULSORB PGC
CHITOSAN FROM MUSHROOM 80 MESH 98% DA
Hydrochloric acid 1N
DEMINERALISED WATER
MICROC. CELLULOSA 200 E460
CALCIUM CARBONATE GRAN.ID PHARMAGNESIA CC TYPE 140
MALTODEXTRINS IT19 (GLUCIDEX) USP
COMPRITOL AND ATO GLYCEROL BEHENATE
SYLOID 244FPNF ID (AMORPHOUS SILICA E551)
KLUCEL NUTRA D
VEG. MAGNESIUM STEARATE VEG. 765130 LIGA E470B
POLYPLASDONE XL
CHAMOMILE E.S. TIT. 0.3% IN APIG.
ANGELICA ARCHANGELICA D.E. 4:1
MINT D.E. 4:1
FENNEL D.E. 4:1
VITAMIN B6 HYDROCHLORIDE PYRID.
VITAMIN B2 BASE RIBOFLAVIN
VITAMIN B1 NITRATE ROCOAT 33% OGM FREE

TABLE 13
The carbon tablet is 550 mg to be administered 2 tablets a day (daily
dose) and contains 100 mg plant carbon, and carrier and excipients.

CARBON TABLET
Batch: NU626
Formulation type: 550 mg capsules
Daily dose: 2 tablets
COMMERCIAL INGREDIENT
Microc. Cellulose 101 E460
Corn starch
Icing sugar
Polyvinylpyrrolidone PVP K30 PI
Plant carbon
Chamomile dry extract ¼
Angelica dry extract ¼
Mint dry extract ¼
Fennel dry extract ¼
Senna d.e. tit. 6-8%.
Vitamin B6 hydrochloride
Vitamin B2 base
Purified water 10%
Compritol E ATO
Vitamin B1 Nitrate Rocoat 33%.

1. A preparation in solid form comprising or, alternatively, consisting of: (i) an activated carbon; and (ii) a chitosan, preferably a plant chitosan from mushrooms, adsorbed onto said activated carbon;
wherein said chitosan has a deacetylation degree from 98% to 100% by weight.

2. The preparation according to the preceding claim, wherein said preparation has:
a specific surface area DFT (Density Functional Theory) according to ISO 15901:2007 from 980 m²/g to 1,300 m²/g; and
a pore volume: from 0.580 cm³/g to 0.750 cm³/g.

3. The preparation according to claim 2, wherein said preparation has:
a specific surface area DFT (Density Functional Theory) according to ISO 15901:2007 from 1,020 m²/g to 1,200 m²/g; and
a pore volume: from 0.590 cm³/g to 0.700 cm³/g.

4. The preparation according to claim 2 or 3, wherein said preparation has:
a specific surface area DFT (Density Functional Theory) according to ISO 15901:2007 from 1,050 m²/g to 1,150 m²/g; and
a pore volume: from 0.620 cm³/g to 0.660 cm³/g.

5. The preparation according to any one of the preceding claims, wherein said preparation comprises (amounts expressed with respect to the total weight of said preparation):
an amount of activated carbon, preferably activated carbon of plant origin, from 85% to 99% by weight; and
an amount of chitosan, preferably plant chitosan from mushrooms with an average molecular weight from 1 kDa to 50 kDa, from 1% to 15% by weight.

6. The preparation according to claim 5, wherein said preparation comprises (amounts expressed with respect to the total weight of said preparation):
an amount of activated carbon, preferably activated carbon of plant origin, from 90% to 98% by weight; and
an amount of chitosan, preferably plant chitosan from mushrooms with an average molecular weight from 1 kDa to 50 kDa, from 2% to 10% by weight.

7. The preparation according to claim 5 or 6, wherein said preparation comprises (amounts expressed with respect to the total weight of said preparation):
an amount of activated carbon, preferably activated carbon of plant origin, from 95% to 97% by weight; and
an amount of chitosan, preferably plant chitosan from mushrooms with an average molecular weight from 1 kDa to 50 kDa, from 3% to 5% by weight.

8. The preparation according to any one of the preceding claims, wherein said preparation is in granulate solid form, wherein said granulate comprises granulate particles having an average particle size distribution such that an amount of from 85% to 100% by weight, preferably from 90% to 99% by weight, passes through a sieve with a nominal aperture of 1.19 mm.

9. A method for preparing a preparation comprising the following steps:
(I) preparing an aqueous solution of a chitosan, preferably plant chitosan from mushrooms, with a deacetylation degree from 98% to 100% by weight;
(II) contacting the aqueous chitosan solution of step (I) with an activated carbon in powder form, to give a chitosan-impregnated activated carbon;
(III) granulating the chitosan-impregnated activated carbon of step (II), to give a granulated impregnated activated carbon;
(IV) drying the granulated impregnated activated carbon of step (III), to give said preparation.

10. The preparation method according to the preceding claim, wherein:
activated carbon in powder form of step (II), preferably an activated carbon of plant origin, comprises powder particles with an average particle size distribution-determined by laser beam diffraction analysis-such that from 55% to 95% by volume, preferably from 65% to 85% by volume, have a size <45 μm; and/or
steps (II) and (III) are at least partially contextual, where the aqueous chitosan solution of step (I) is contacted with activated carbon in powder form pre-loaded in a granulation device equipped with an impeller that has already been activated in rotation.

11. A composition in solid form comprising the preparation according to any one of claims 1-8, or the preparation obtained by the method according to any one of claims 9-10, and at least one physiologically and/or pharmaceutically acceptable excipient; wherein:
said composition is in tablet solid form and includes:
said preparation in an amount from 5% to 40%, preferably from 10% to 30%;
diluents, preferably starch (more preferably corn starch) and/or maltodextrins, in an amount from 10% to 60%;
anti-caking agents, preferably glycerol behenate, silicon salts (preferably amorphous silica) and/or magnesium stearate, in an amount from 0.5% to 5%;
a disintegrant, preferably sodium carboxymethyl cellulose, in an amount from 0.5% to 4%; and
a binder, preferably microcrystalline cellulose, in an amount from 10% to 45%; or said composition is in capsule solid form and includes:
said preparation in an amount from 70% to 99.8%;
an anti-caking agent, preferably magnesium stearate, in an amount from 0.1% to 5%; or a diluent, preferably maltodextrin, in an amount from 3% to 25%.

12. A composition in solid form comprising the preparation according to claim 11, and at least one physiologically and/or pharmaceutically acceptable excipient; wherein:
said composition is in tablet solid form and includes:
said preparation in an amount from 15% to 25%, preferably from 16% to 22%;
diluents, preferably starch (more preferably corn starch) and/or maltodextrins, in an amount from 35% to 55%, preferably from 40% to 50%;
anti-caking agents, preferably glycerol behenate, silicon salts (preferably amorphous silica) and/or magnesium stearate, in an amount from 2% to 4.5%, preferably from 3% to 4%;
a disintegrant, preferably sodium carboxymethyl cellulose, in an amount from 1% to 3%, preferably from 1.5% to 2.5%; and
a binder, preferably microcrystalline cellulose, in an amount from 20% to 40%, preferably from 25% to 35%; or said composition is in capsule solid form and includes:
said preparation in an amount from 75% to 99.5%, preferably from 80% to 99%;
an anti-caking agent, preferably magnesium stearate, in an amount from 0.5% to 2%; or a diluent, preferably maltodextrin, in an amount from 5% to 20%, preferably from 7% to 17%.

13. The composition according to any preceding claim, wherein said composition is for use in a method of treatment, preventative or curative, of a gastrointestinal disorder, or discomfort, or symptom, or disease associated with the presence or production of gas in the stomach and/or intestines of a subject;
preferably said composition being orally administered to said subject.

14. The composition for use according to any preceding claim, wherein said gastrointestinal disorder, discomfort, symptom, or disease is selected from the group comprising or, alternatively, consisting of: belching, flatulence, distension or swelling of the abdomen, abdominal pain, and meteorism.

15. The composition according to claim 12, wherein said composition is for use in a method of treatment, preventive or curative, of a disorder, or discomfort, or a symptom, or a disease associated with hypercholesterolaemia in a subject.
